(19) United States
(12) Patent Application Publication
(10) Pub. No.: US 2023/0080010 A1
(43) Pub. Date: Mar. 16, 2023

(54) NEBULIZER SYSTEM FOR A MOTOR VEHICLE

(71) Applicant: VALEO SYSTEMES THERMIQUES, La Verriere (FR)

(72) Inventors: Clement ESCULIER, La Verriere (FR); Vincent FEUILLARD, La Verriere (FR)

(73) Assignee: VALEO SYSTEMES THERMIQUES, La Verriere (FR)

(21) Appl. No.: 17/796,083

(22) PCT Filed: Jan. 28, 2021
(86) PCT No.: PCT/FR2021/050156

§ 371 Date: Jul. 28, 2022

(30) Foreign Application Priority Data
Jan. 29, 2020 (FR) FR2000858

(51) Int. Cl. B05B 17/06; B60H 3/02; C02F 1/32

(52) U.S. Cl. CPC B05B 17/0615; B60H 3/022; C02F 1/325

(57) ABSTRACT

The invention relates to a nebulizer system for a motor vehicle, including at least one reservoir for storing a liquid, a nebulizer component including an acoustic wave emission device configured such that the liquid forms a mist of droplets of the liquid, the mist being intended to enter a passenger compartment of the motor vehicle, a light-emitting device configured to emit radiation in the ultraviolet spectrum, and a conduit for delivering the liquid to at least one inlet of the nebulizer component. The light-emitting device is oriented in the nebulizer system so as to irradiate the liquid circulating in the conduit for delivering the liquid, when the nebulizer system is in the operating state.

### CROSS-REFERENCE TO RELATED APPLICATIONS

This application is filed under 35 U.S.C. § 371 U.S. National Phase of International Application No. PCT/FR2021/050156 filed Jan. 28, 2021 (published as WO2021152261), which claims priority benefit to French application No. 2000858 filed on Jan. 29, 2020, the disclosures of which are herein incorporated by reference in their entirety.

### TECHNICAL FIELD

The subject of the invention is a nebulizer system for a motor vehicle.

### BACKGROUND OF THE INVENTION

In a known manner, a nebulizer system comprises a reservoir of liquid water and a device for emitting acoustic waves configured such that the water coming from the reservoir forms a mist of water droplets.

Such a nebulizer system makes it possible to cool an air flow in which the mist of water is sprayed.

In a motor vehicle, even one provided with a ventilation and/or air-conditioning device, it is not uncommon for the users of the vehicle to suffer under heat in the vehicle interior, in particular the rear seat passengers, who are farther away from the air vents of the ventilation and/or air-conditioning device.

As a result, it is advantageous to make use of a nebulizer system, since the mist of water rapidly cools the air in the vehicle interior, providing a sensation of immediate cold while still rehumidifying it.

Because the water coming from the nebulizer system comes in direct contact with the skin of the users of the vehicle, it is essential to ensure that the nebulizer system is hygienic.

For the other part, the nebulizer system must be as compact as possible because it constitutes an additional element of the vehicle that it occupies.

The objective of the invention is to propose a nebulizer system that is not dangerous to the health of the users of the motor vehicle while still being compact.

### BRIEF SUMMARY OF THE INVENTION

To that end, a subject of the invention is a nebulizer system for a motor vehicle, comprising at least one reservoir for storing a liquid, a nebulizing component having a device for emitting acoustic waves configured such that said liquid forms a mist of droplets of said liquid, the mist being intended to enter a vehicle interior of the motor vehicle, an electroluminescent device configured to emit radiation in the ultraviolet spectrum, and a duct for conveying the liquid toward at least one inlet of a nebulizing component having, the electroluminescent device being oriented so as to irradiate the liquid circulating in the duct for conveying the liquid, when the nebulizer system is operating.

Thus, by virtue of the electroluminescent device, the bacteria that are able to proliferate in the liquid circulating in the nebulizer system are effectively neutralized, without it being necessary to make use of a filter, the very shape of the support ensuring that the nebulizer system is more compact.

In addition, since the ultraviolet radiation penetrates the liquid, preferably water, having a high absorption coefficient, it essentially diffuses into the liquid, thereby reducing the risks of exposing the users of the motor vehicle to the radiation.

According to another aspect, the ultraviolet spectrum comprises wavelengths in a range of between 200 nm and 290 nm.

According to another aspect, the system comprises a housing of the electroluminescent device provided with a housing channel for the liquid to pass through, the housing channel being integral with the duct for conveying the liquid.

In another aspect, the system comprises at least one baffle for guiding the liquid toward the housing.

According to another aspect, the duct for conveying the liquid comprises an upper wall and a base wall which are spaced apart from one another, the upper wall comprising at least one inlet orifice for entry of the liquid into the duct for conveying the liquid.

According to another aspect, the system comprises a sphere for confining the ultraviolet radiation emitted by the electroluminescent device.

According to another aspect, the system comprises a sphere channel for the liquid to pass through in the sphere.

According to another aspect, the sphere channel passes through the center of the sphere.

Another subject of the invention is a ventilation, heating and/or air-conditioning device for a motor vehicle, comprising a nebulizer system as described above.

### BRIEF DESCRIPTION OF THE DRAWINGS

Other features, details and advantages of the invention will become apparent upon reading the detailed description below, and upon analyzing the appended drawings, in which:

FIG. 1 illustrates a schematic view of a partial longitudinal section of a nebulizer system according to a first embodiment of the present invention,

FIG. 2 illustrates a schematic view of a partial longitudinal section of a nebulizer system according to a second embodiment of the present invention,

FIG. 3 illustrates a partial longitudinal section of a nebulizer system according to a third embodiment of the present invention, and

FIG. 4 illustrates a partial longitudinal section of the system from FIG. 3.

### DETAILED DESCRIPTION OF THE INVENTION

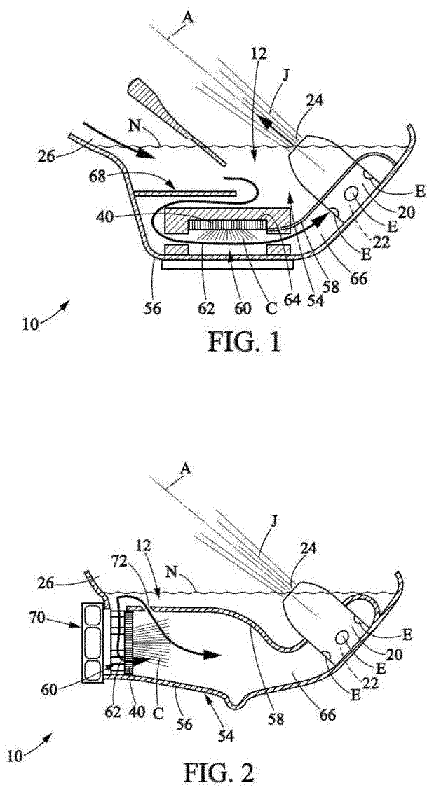

A subject of the invention is a nebulizer system 10 for a motor vehicle, as shown for example in FIGS. 1 to 3.

The nebulizer system 10 makes it possible to cool an air flow intended for the interior of the motor vehicle, as will be described in more detail. The nebulizer system 10 is, for example, intended to be incorporated in a central console arranged in the interior of the motor vehicle.

As can be seen in the figures, the nebulizer system 10 comprises a reservoir 12 for storing a fluid, preferably liquid water, that is set out below, otherwise referred to as tank.

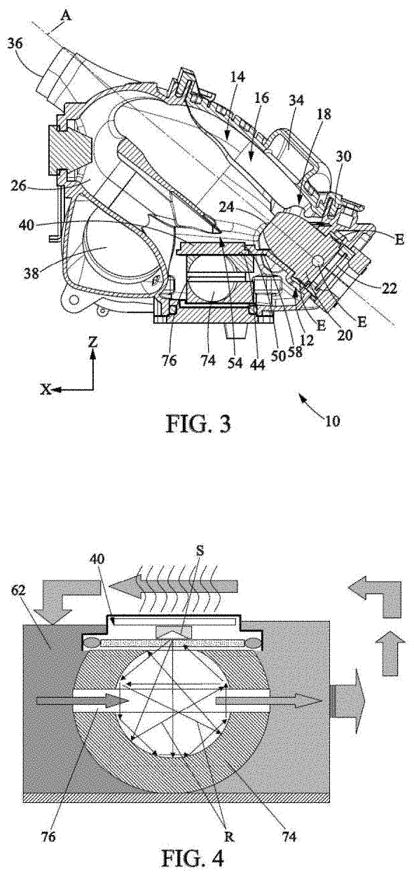

The nebulizer system 10 preferably comprises a nebulizer enclosure 14, illustrated in FIG. 3. Here, the nebulizer enclosure 14 is realized in the form of an elongate hollow body extending along a longitudinal axis A.

In the illustrated embodiments, the longitudinal axis A is inclined with respect to a vertical direction Z and with respect to a horizontal direction X.

As is apparent in particular from FIG. 3, the nebulizer enclosure 14 has at least two parts arranged one after the other in the direction of the longitudinal axis A, specifically a nebulizer chamber 16 and a nebulizer tube 18, the nebulizer tube 18 leading into the nebulizer chamber 16.

As is apparent from FIGS. 1 to 3, the nebulizer system 10 has a nebulizing component realized here in the form of a nebulizer nozzle 20 provided with a device for emitting acoustic waves transmitted into the liquid, which device is configured such that the surface of the liquid coming from the nebulizer nozzle 20 generates a mist of droplets of the liquid, this mist being intended to enter an interior of the motor vehicle.

It will be noted that the longitudinal axis A also corresponds to the direction in which the jet J of water from which the mist is ejected from the nebulizer nozzle 20 into the nebulizer chamber 16.

The nebulizer nozzle 20 is arranged at least partially inside the nebulizer enclosure 14. The nebulizer nozzle 20 has a lateral wall delimiting an interior volume which is able to contain the liquid to be nebulized. The internal cross section of this lateral wall narrows gradually in the direction of an outlet orifice 24 for the liquid, making it possible to form an acoustic wave concentrator.

A piezoelectric (ceramic) element 22 is arranged on the opposite side to an outlet orifice 24 for the liquid.

The piezoelectric element 22 is able to emit acoustic waves into the liquid to be sprayed, thereby making it possible to generate a mist of liquid droplets when the nebulizer nozzle 20 is filled by the latter and when the piezoelectric element 22 emits acoustic waves of a suitable frequency and intensity. The piezoelectric element 22 will preferably be able to emit ultrasound at a frequency of between 1 MHz and 3 MHz, in particular between 1.7 MHz and 2.4 MHz.

For example, the diameter of the droplets present in the mist is less than 10 μm.

The nebulizer nozzle 20 also has at least one intake orifice E for the liquid to be nebulized which allows the introduction of the liquid to be nebulized into the interior volume of the nebulizer nozzle 20, in fluidic communication with the reservoir 12.

As is apparent in FIGS. 1 to 3, the nebulizer system 10 also comprises a duct 26 for returning the liquid to the reservoir 12. In the embodiment illustrated in FIG. 3, the duct 26 continues the nebulizer chamber 20 with formation of an almost 180° curve. The curved shape of the duct 26 ensures improved compactness of the nebulizer system 10.

The nebulizer system 10 can also comprise an inlet channel 30 for water intended to accommodate a cartridge that contains liquid and thereby constitutes an additional, advantageously removable reservoir of the system 10, as illustrated in FIG. 3.

As can be seen in FIG. 3, the nebulizer system 10 further has an inlet duct 34 for air and also at least one outlet duct 36 for the mist toward the interior of the vehicle, and a fan 38 forcing the movement of the air.

Thus, the air enters the nebulizer enclosure 14 at the nebulizer chamber 16, where the air and the nebulized liquid mix before the mixture leaves the nebulizer system 10 via the outlet duct 36 for the mist, where it circulates to the interior of the vehicle.

The nebulizer system 10 also comprises an electroluminescent device 40.

The electroluminescent device 40 has an ultraviolet radiation source with emitted wavelengths within the ultraviolet C spectrum, between 200 nm and 300 nm and, preferably, between 200 nm and 280 nm. The ultraviolet C radiation makes it possible, in a known manner, to kill bacteria. The radiation source S can be seen in FIG. 4.

Advantageously, the electroluminescent device 40 consists of a photolysis reactor with light-emitting diode(s).

The ultraviolet radiation source S, and also an associated electronic circuit, are embedded in a resin 44 bordered by a seal. The electroluminescent device 40 comprises a base 50, preferably made of stainless steel, arranged on the resin 44.

As can be seen from the figures, the reservoir 12 comprises an intermediate portion 54, arranged between the return duct 26 and the nebulizer nozzle 20.

The intermediate portion 54 comprises a planar base wall 56 and an upper wall 58 arranged above the base wall 56 so as to force the water to pass between the electroluminescent device 40 and the nebulizer nozzle 20, as will be described in detail with reference to each of the embodiments.

It will be noted that, during operation, the resin 44 and the base 50 are submerged in the reservoir 12, a water level in the reservoir 12 being referenced N, as will also be described in detail later on.

Thus, during operation, the cartridge containing liquid is secured to the inlet channel 30, and the water level N reaches the nebulizer tube 18, a part of the duct 26 for the return of the liquid to the reservoir 12, and the intermediate portion 54.

It will be noted that this configuration makes it possible not to equip the nebulizer system 10 with a water circulation pump.

The first embodiment, illustrated in FIG. 1, will now be described in detail.

In FIG. 1, the intermediate portion 54 comprises a housing 60 of the electroluminescent device 40 provided with a housing channel 62 for water to pass through. The ultraviolet radiation source embedded in the resin 44 is fitted in a top part 64 of the housing 60 of the electroluminescent device 40. The ultraviolet radiation source is oriented toward the housing channel 62 for water to pass through, such that the water circulating in the housing channel 62 for water to pass through is irradiated as it traverses an irradiation cone C.

As can be seen in this figure, the housing 60 of the electroluminescent device 40 is placed on the base wall 56. The upper wall 58 extends from the housing 60 of the electroluminescent device 40, and then around the nebulizer nozzle 20 to beyond the nebulizer nozzle 20, thereby ensuring that the water is conveyed from the housing channel 62 for water to pass through to the inlet orifice E of the nebulizer nozzle 20, while still forcing the water to traverse the housing channel 62 for water to pass through before arriving at the nebulizer nozzle 20. In other words, this configuration forms a duct 66 for conveying the water to the nebulizer nozzle 20 which ensures that all the water present in the nebulizer system 10 is treated by the ultraviolet radiation.

The nebulizer system 10 also comprises at least one baffle in the intermediate portion 54 of the reservoir 12. FIG. 1 illustrates a single baffle 68 which ensures that the water moves in an S-shaped current above the top part 64 of the housing 60 of the electroluminescent device 40. This current makes it possible to effectively cool the electroluminescent device 40, and in particular the stainless steel base 50.

The second embodiment, illustrated in FIG. 2, will now be described in detail.

In FIG. 2, the intermediate portion 54 comprises a housing 60 of the electroluminescent device 40 provided with a housing channel 62 for water to pass through. The housing 60 of the electroluminescent device 40 is secured to a connector 70 forming the transverse wall of the reservoir 12 between the return duct 26 for returning the liquid to the reservoir 12 and the base wall 56, arranged such that the electroluminescent device 40 is arranged substantially vertically in the reservoir 12. The housing channel 62 for water to pass through ensures effective cooling of the electroluminescent device 40, and in particular of the stainless steel base 50.

The upper wall 58 extends from the housing 60 of the electroluminescent device 40, and then around the nebulizer nozzle 20 to beyond the nebulizer nozzle 20, a space between the base wall 56 and upper wall 58 forming a duct 66 for conveying water to the nebulizer nozzle 20.

An orifice 72, which ensures that water is conveyed between the return duct 26 for returning the liquid to the reservoir 12 and the nebulizer nozzle 20, is made in the upper wall 58.

The electroluminescent device 40 is oriented toward the duct 66 for conveying the water, such that the water circulating in the duct 66 for conveying the water is irradiated as it traverses the irradiation cone C.

It will be noted that the duct 66 for conveying the water ensures that all the water present in the nebulizer system 10 is treated by the ultraviolet radiation.

The third embodiment, illustrated in FIGS. 3 and 4, will now be described in detail.

As can be seen in these figures, the intermediate portion 54 is provided with a sphere 74, preferably made of white Teflon (which has a UV reflection coefficient of 97%), provided with a sphere channel 76 for water passing through the center of the sphere 74. The sphere 74 ensures convergence of the ultraviolet rays R toward the center of the sphere 74, thereby improving the irradiation power of the electroluminescent device 40, as illustrated in FIG. 4. The ultraviolet radiation source embedded in the resin 44 is arranged on the sphere 74 and oriented toward the sphere channel 76 for water to pass through, such that the water circulating in the sphere channel 76 is irradiated as it traverses the sphere 74. The sphere 74 also makes it possible to improve the safety of the nebulizer system 10 by way of its confinement of ultraviolet C rays.

As can be seen in FIG. 4, the cooling of the electroluminescent device 40 is optimized by way of its immersion in water.

As can be seen in FIG. 3, the upper wall 58 extends from the sphere 74, and then around the nebulizer nozzle 20 to beyond the nebulizer nozzle 20, thereby ensuring that the water is conveyed from the housing channel 62 for water to pass through to the inlet orifice E of the nebulizer nozzle 20, while still forcing the water to traverse the housing channel 62 before arriving at the nebulizer nozzle 20. In other words, this configuration forms a duct 66 for conveying the water to the nebulizer nozzle 20, which ensures that all the water present in the nebulizer system 10 is treated by the ultraviolet radiation.

By virtue of the antibacterial treatment, bacteria cannot proliferate in the nebulizer system 10, thereby ensuring that the nebulizer system 10 is always hygienic, whether the nebulizer system 10 is operating or not. In addition, since the electroluminescent device 40 directly irradiates the liquid, the propagation of the ultraviolet radiation is limited to the nebulizer system 10, thereby ensuring the safety of the users of the vehicle. The immersion of the electroluminescent device 40 also ensures effective cooling of the radiation source(s). The arrangement of the duct 66 for conveying the water also makes it possible to treat all the water before it enters the nebulizer nozzle 20.

What is claimed is:

1. A nebulizer system for a motor vehicle, comprising:
at least one reservoir for storing a liquid,
nebulizing component having a device for emitting acoustic waves that is configured such that said liquid forms a mist of droplets of said liquid, the mist being intended to enter an interior of the motor vehicle,
an electroluminescent device configured to emit radiation in the ultraviolet spectrum, and
a duct for conveying the liquid toward at least one inlet of the nebulizing component,
the electroluminescent device being oriented in the nebulizer system so as to irradiate the liquid circulating in the duct for conveying the liquid, when the nebulizer system is in operation.

2. The nebulizer system as claimed in claim 1, wherein the ultraviolet spectrum comprises wavelengths of between 200 nm and 280 nm.

3. The nebulizer system as claimed in claim 1, including a housing of the electroluminescent device provided with a housing channel for the liquid to pass through, the housing channel being integral with the duct for conveying the liquid.

4. The nebulizer system as claimed in claim 3, including at least one baffle for guiding the liquid toward the housing of the electroluminescent device.

5. The nebulizer system as claimed in claim 1, wherein the duct for conveying the liquid comprises an upper wall and a base wall which are spaced apart from one other, the upper wall including at least one inlet orifice for entry of the liquid into the duct for conveying the liquid.

6. The nebulizer system as claimed in claim 1, including a sphere for confinement of the ultraviolet radiation emitted by the electroluminescent device.

7. The nebulizer system as claimed in claim 6, including a sphere channel in the sphere for the liquid to pass through.

8. The nebulizer system as claimed in claim 7, wherein the wherein the sphere channel passes through the center of the sphere.

9. A ventilation, heating and/or air-conditioning device for a motor vehicle, including
a nebulizer system for a motor vehicle, including:
at least one reservoir for storing a liquid,
nebulizing component having a device for emitting acoustic waves that is configured such that said liquid forms a mist of droplets of said liquid, the mist being intended to enter an interior of the motor vehicle,
an electroluminescent device configured to emit radiation in the ultraviolet spectrum, and
a duct for conveying the liquid toward at least one inlet of the nebulizing component,
the electroluminescent device being oriented in the nebulizer system so as to irradiate the liquid circulating in the duct for conveying the liquid, when the nebulizer system is in operation.
